SEQUENCE LISTING (8 sequences)

SEQ ID NO: 1 - DNA, 769 bp, Artificial Sequence
  expression construct
ggatcccacaatgaagctttccaccatccttttcacagcctgcgctaccctggctgccgccctcccttcccccatcactccttctgaggccgcagttctccagaagcgaggcggcggcgacattcagatgactcagtctccctcttctctgtctgcttctgtgggtgaccgagtgaccattacctgtcgagcttctcaggacgtgaacactgctgttgcttggtatcagcagaagcctggaaaggctcctaagctgctgatctactctgcctctttcctgtactctggcgtgccttctcgattttctggctctcgatctggaaccgacttcaccctgaccatttcttctctgcagcctgaggactttgctacctactactgtcagcagcattacaccacccctcctacttttggacagggcaccaaggttgagattaagcgaaccgtggctgctccttctgtgttcattttccccccctctgacgagcagctgaagtctggaactgcttctgttgtgtgcctgctgaacaacttttacccccgagaggctaaggttcagtggaaggtggacaacgctctgcagtctggaaactctcaggagtctgttactgagcaggactctaaggactcgacctactctctctcttctaccctgaccctgtctaaggctgactacgagaagcataaggtgtacgcttgtgaggttacccatcagggactgtcctctcccgtgaccaagtcttttaaccgaggcgagtgctaacctagg

SEQ ID NO: 2 - DNA, 1482 bp, Artificial Sequence
  expression construct
gcaacggatcccacaatgaagctttccaccatccttttcacagcctgcgctaccctggctgccgccctcccttcccccatcactccttctgaggccgcagttctccagaagcgaggcggcggcgaggttcagctggttgagtctggtggaggactggttcagcctggtggatctctgcgactgtcttgtgctgcttctggcttcaacatcaaggacacctacattcattgggtccgacaggctcccggaaagggactggagtgggttgcccgaatctaccctaccaacggctacactcgatacgctgactctgtgaagggacgattcaccatttctgccgacacctctaagaacactgcctacctgcagatgaactctctgcgagctgaggacactgctgtgtactactgttctcgatggggaggtgacggtttttacgccatggactactggggacagggaactctggtgaccgtttcttctgcttctaccaagggaccttctgtgtttcctctggccccctcttctaagtctacctctggtggaactgctgctctgggatgtctggtgaaggactactttcctgagcctgtgactgtgtcttggaactctggcgctctgacttctggtgttcacaccttccctgctgttctgcagtcctctggactgtactctctctcttctgtggtgaccgtgccttcttcttctctgggaacccagacctacatctgtaacgtgaaccacaagccctctaacactaaggtggacaagcgagtggagcctaagtcttgtgacaagacccatacctgtcccccttgtcctgctcctgagctgctgggaggaccctctgtttttctgttcccccccaagcctaaggacaccctgatgatttctcgaacccctgaggtgacctgtgttgtggtggacgtttctcatgaggaccctgaggtgaagtttaactggtacgtggacggtgttgaggttcacaacgctaagactaagccccgagaggagcagtacaactctacttaccgagtggtgtctgtgctgactgttctgcatcaggactggctgaacggaaaggaatacaagtgtaaggtctccaacaaggctctgcctgctcctattgaaaagaccatctctaaggctaagggacagcccagagagcctcaggtttacactctgcccccttcccgagaggagatgaccaagaaccaggtgtccctgacttgtctggtcaagggattctacccctctgacattgctgttgagtgggagtctaacggacagcctgagaacaactacaagaccacccctcctgttctggactctgacggctctttcttcctgtactctaagctgaccgtggacaagtctcgatggcagcagggaaacgtgttctcttgttccgtgatgcatgaggctctgcacaaccactacacccagaagtctctgtctctgtctcccggcaagtaacctagg

SEQ ID NO: 3 - protein, 534 aa, Yarrowia lipolytica
MHFSNFLLGALAATAAAKNTYQINKYGSPLTNQKRSLSENSVVQLDTVGVRSIRDEPAPRDAALMKRQTATLPLKNLVTYYEAEVKIGTPAQTVKLLIDTGSSDIWVIGSGNPDCGSAQDAQRDPNIIDCSISGTFDTSKSSSWSQNQTDFFIQYGDQTAAEGGWGTDTFAFGNTNVSGLSIAVASKTNSSNGVMGIGLAGLESTITYRGNDQISGNPYENLPMKMKAEGLIKANAYSLWLNNLSSDSGNVLFGGVDYAKIDGDLFTVKLVNPQRSVSSKPIAFYVGLDSVSITDVKGVSGFITKQPVPALLDSGTTLTYLPQDAFNYVVRAMGATYDPQNGYVCPCKNGYSGHLDYNFSGANISVPLYQLTYPIQLQSQSGRVVNAQFRNGDDACLLLMQASQDHVILGDSFLRAAYVVYNLDSYEVSMGQTKYGVTDTNIVEIDSNGVKNANPAPEYSSSFTNVNSETTILRGAPGSADSNPSTTLSGGLVAGSSASSGSSGDGKGKNNAAGLELSIVGLAVAVVMASFGLM

SEQ ID NO: 4 - protein, 727 aa, Yarrowia lipolytic
MKSLLLSLLAVPATAQLLLDLQGHDTTDPRHQSQNHLTKRKTVEQDLVQKFAYYEATVSVGTPGQQIKLLLDTGSSDMWVLGQNVNCGGGGIFSQGIDCTQSGVFDTSKSSTYHKNESIPFDIKYSDGSESKGFYGTDNLGLGGTTLNDFTFAVADSASDGQAVLGIGPIENEQSLYTDNPVAYANLPLALMMDGVTKSSAFSLWLNDKDALKGSILFGGYDRAKVDGDLFTVPIVNLNPGGQGRSREYNVGLDSITVGGKSVGSSTPALLDSGTTLCNLPQEMVDAILAQFSGVSNSLRAGYYTKCSNVPQGSIDFVFSGNKLTVELADLMKPLENPDGSKITTDGEQACGILVTSNTDSHSISDSVVLGASFLRSAYVVYDRDAQKIMMGKAKYGVSSSDIVELKDGSSEGVASAESSAESAAATASSSAGASSGASSSGASSAGASSDSSASASASASASASASASATATSEGDSGQGVSTMAVVVRPSDTRLSAYITNIVVTPSATPALSTVSVVVRPSDTRLSAYITEIVVTPTAVPFSTVVSTTAIESTEIVTVTSCSDGKCEHAKSTVYKIVDATVTQTIWSCEGDESATWAPAPTPEPQNTQVQNTPAYQAPSAPPVFLPVWGTQSDGETVTHTETAFFNPQTYTGPPAQPTGASGGNGGNNGGNNGGNNGGNNGGNGGNDNNGGSSSHSSGITQANGASSISPMISLVVLLLSFLIWA

SEQ ID NO: 5 - DNA, 757 bp, Artificial Sequence
  nucleotide sequence encoding light chain
cacaatgaagctttccaccatccttttcacagcctgcgctaccctggctgccgccctcccttcccccatcactccttctgaggccgcagttctccagaagcgaggcggcggcgacattcagatgactcagtctccctcttctctgtctgcttctgtgggtgaccgagtgaccattacctgtcgagcttctcaggacgtgaacactgctgttgcttggtatcagcagaagcctggaaaggctcctaagctgctgatctactctgcctctttcctgtactctggcgtgccttctcgattttctggctctcgatctggaaccgacttcaccctgaccatttcttctctgcagcctgaggactttgctacctactactgtcagcagcattacaccacccctcctacttttggacagggcaccaaggttgagattaagcgaaccgtggctgctccttctgtgttcattttccccccctctgacgagcagctgaagtctggaactgcttctgttgtgtgcctgctgaacaacttttacccccgagaggctaaggttcagtggaaggtggacaacgctctgcagtctggaaactctcaggagtctgttactgagcaggactctaaggactcgacctactctctctcttctaccctgaccctgtctaaggctgactacgagaagcataaggtgtacgcttgtgaggttacccatcagggactgtcctctcccgtgaccaagtcttttaaccgaggcgagtgctaa

SEQ ID NO: 6 - protein, 250 aa, Artificial Sequence
  light chain sequence
MKLSTILFTACATLAAALPSPITPSEAAVLQKRGGGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - DNA, 1465 bp, Artificial Sequence
  Nucleotide sequence encoding heavy chain
cacaatgaagctttccaccatccttttcacagcctgcgctaccctggctgccgccctcccttcccccatcactccttctgaggccgcagttctccagaagcgaggcggcggcgaggttcagctggttgagtctggtggaggactggttcagcctggtggatctctgcgactgtcttgtgctgcttctggcttcaacatcaaggacacctacattcattgggtccgacaggctcccggaaagggactggagtgggttgcccgaatctaccctaccaacggctacactcgatacgctgactctgtgaagggacgattcaccatttctgccgacacctctaagaacactgcctacctgcagatgaactctctgcgagctgaggacactgctgtgtactactgttctcgatggggaggtgacggtttttacgccatggactactggggacagggaactctggtgaccgtttcttctgcttctaccaagggaccttctgtgtttcctctggccccctcttctaagtctacctctggtggaactgctgctctgggatgtctggtgaaggactactttcctgagcctgtgactgtgtcttggaactctggcgctctgacttctggtgttcacaccttccctgctgttctgcagtcctctggactgtactctctctcttctgtggtgaccgtgccttcttcttctctgggaacccagacctacatctgtaacgtgaaccacaagccctctaacactaaggtggacaagaaggtggagcctaagtcttgtgacaagacccatacctgtcccccttgtcctgctcctgagctgctgggaggaccctctgtttttctgttcccccccaagcctaaggacaccctgatgatttctcgaacccctgaggtgacctgtgttgtggtggacgtttctcatgaggaccctgaggtgaagtttaactggtacgtggacggtgttgaggttcacaacgctaagactaagccccgagaggagcagtacaactctacttaccgagtggtgtctgtgctgactgttctgcatcaggactggctgaacggaaaggaatacaagtgtaaggtctccaacaaggctctgcctgctcctattgaaaagaccatctctaaggctaagggacagcccagagagcctcaggtttacactctgcccccttcccgagaggagatgaccaagaaccaggtgtccctgacttgtctggtcaagggattctacccctctgacattgctgttgagtgggagtctaacggacagcctgagaacaactacaagaccacccctcctgttctggactctgacggctctttcttcctgtactctaagctgaccgtggacaagtctcgatggcagcagggaaacgtgttctcttgttccgtgatgcatgaggctctgcacaaccactacacccagaagtctctgtctctgtctcccggcaagtaa

SEQ ID NO: 8 - protein, 486 aa, Artificial Sequence
  Heavy chain sequence
MKLSTILFTACATLAAALPSPITPSEAAVLQKRGGGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2015/0031081 A1
(43) Pub. Date: Jan. 29, 2015

(54) METHODS AND MATERIALS FOR REDUCING DEGRADATION OF RECOMBINANT PROTEINS

(71) Applicant: Oxyrane UK Limited, Manchester (GB)

(72) Inventors: Wouter Vervecken, Gent-Ledeberg (BE); Stefan Ryckaert, Sint-Amandsberg (BE)

(21) Appl. No.: 14/369,324

(22) PCT Filed: Dec. 28, 2012
(86) PCT No.: PCT/IB2012/003077

§ 371 Date: Jun. 27, 2014

Related U.S. Application Data
(60) Provisional application No. 61/581,859, filed on Dec. 30, 2011.

(51) Int. Cl. C12N 15/80; C07K 16/22; C07K 16/40

(52) U.S. Cl. CPC C12N 15/80; C07K 16/40; C07K 16/22; C07K 2317/14
USPC 435/69.6; 435/254.11

(57) ABSTRACT

Described herein are methods and materials for reducing degradation of recombinant proteins in fungal cells such as Yarrowia.

### CROSS-REFERENCE TO RELATED APPLICATIONS

This application claims the benefit of priority to U.S. Provisional Application Ser. No. 61/581,859, filed on Dec. 30, 2011, the contents of which are incorporated herein by reference in its entirety.

### TECHNICAL FIELD

This invention relates to methods and materials for reducing degradation of recombinant proteins in fungal cells, and more particularly, to genetically engineered Yarrowia cells with deficiencies in two different yapsin peptidase activities.

### BACKGROUND

High performance expression systems are required to produce most biopharmaceuticals (e.g., recombinant proteins) currently under development. Yeast-based expression systems combine the ease of genetic manipulation and fermentation of a microbial organism with the capability to secrete and to modify proteins. However, the recombinant proteins are often degraded by intracellular proteases as well as extracellular proteases. Thus, there is a need for a yeast based expression system with reduced degradation of the recombinant proteins.

### SUMMARY

This document is based at least in part on the discovery that degradation of recombinant proteins is reduced in Yarrowia cells that have deficiencies in two different yapsin peptidase activities, YPS1 protein (pYPS1) and YPS2 protein (pYPS2). Genetically engineered Yarrowia strains described herein are useful for producing undegraded recombinant proteins (e.g., antibodies).

In one aspect, this document features an isolated Yarrowia cell (e.g., a Yarrowia lipolytica cell) genetically engineered to comprise a deficiency in pYPS1 activity and a deficiency in pYPS2 activity. In some embodiments, the cell does not produce detectable levels of a functional pYPS1 or a functional pYPS2. In some embodiments, the cell does not produce detectable mRNA molecules encoding a functional pYPS1 and a functional pYPS2. In some embodiments, the YPS1 and YPS2 genes are disrupted in the cell. In some embodiments, the YPS1 and YPS2 open reading frames are deleted.

In another aspect, this document features a substantially pure culture of Yarrowia lipolytica cells, a substantial number of which are genetically engineered to comprise a deficiency in pYPS1 activity and a deficiency in pYPS2 activity.

This document also features a method for reducing degradation of a target protein produced in Yarrowia. The method includes expressing a nucleic acid encoding the target protein in a Yarrowia cell described herein.

In another aspect, this document features a method for producing a target protein. The method includes providing a Yarrowia cell genetically engineered to comprise a deficiency in pYPS1 activity, a deficiency in pYPS2 activity, and a nucleic acid encoding the target protein; and b) culturing the cell under conditions such that the cell produces the target protein.

Any of the cells described herein further can be deficient in OCH 1 activity.

Any of the cells described herein further can include a nucleic acid encoding an alpha-1,2 mannosidase The alpha-1,2 mannosidase can include a targeting sequence to target the alpha-1,2 mannosidase to an intracellular compartment.

Any of the cells described herein further can be deficient in ALG3 activity.

Any of the cells described herein further can include a nucleic acid encoding an alpha-1,3-glucosyltransferase.

Any of the cells described herein further can include a nucleic acid encoding the alpha and beta subunits of a glucosidase.

Any of the cells described herein further can include a nucleic acid encoding a GlcNAc-transferase I. The GlcNAc-transferase I can include a targeting sequence to target the GlcNAc-transferase I to an intracellular compartment.

Any of the cells described herein further can include a nucleic acid encoding a GlcNAc-transferase II. The GlcNAc-transferase II can include a targeting sequence to target the GlcNAc-transferase II to an intracellular compartment.

Any of the cells described herein further can include a nucleic acid encoding a galactosyltransferase. The galactosyltransferase can include a targeting sequence to target the galactosyltransferase to the Golgi apparatus.

Any of the cells described herein further can include a nucleic acid encoding a target protein (e.g., a lysosomal protein, a pathogen protein, a growth factor, a cytokine, a chemokine, one or two polypeptide chains of an antibody or antigen-binding fragment thereof, or a fusion protein). The antibody can be selected from the group consisting of an antibody that binds vascular endothelial growth factor (VEGF), an antibody that binds to epidermal growth factor receptor (EGFR), an antibody that binds to CD3, an antibody that binds to tumor necrosis factor (TNF), an antibody that binds to TNF receptor, an antibody that binds to CD20, an antibody that binds to glycoprotein IIa/IIb receptor, an antibody that binds to IL2-receptor, an antibody that binds to CD52, an antibody that binds to CD11a, and an antibody that binds to HER2. The antigen-binding fragment can be selected from the group consisting of Fab, F(ab′)2, Fv, and single chain Fv (scFv) fragments.

This document also features an isolated Yarrowia cell genetically engineered to comprise (i) a deficiency in pYPS1 activity and (ii) a deficiency in pYPS2 activity; and one or more of (iii) a deficiency in ALG3 activity, (iv) a deficiency in OCH1 activity, (v) a nucleic acid encoding an alpha-1,2 mannosidase, (vi) a nucleic acid encoding a GlcNAc-transferase I, (vii) a nucleic acid encoding a GlcNAc-transferase II, (viii) a nucleic acid encoding a mannosidase II, (ix) a nucleic acid encoding an α-1,3-glucosyltransferase, (x) a nucleic acid encoding a galactosyltransferase, and (xi) a nucleic acid encoding the α and β subunits of a glucosidase. For example, such a cell can include (i) a deficiency in pYPS1 activity; (ii) a deficiency in pYPS2 activity; (iii) a deficiency in ALG3 activity; (iv) a deficiency in OCH1 activity; (v) a nucleic acid encoding an alpha-1,2mannosidase; (vi) a nucleic acid encoding a GlcNAc-transferase I; (vii) a nucleic acid encoding a GlcNAc-transferase II; (viii) a nucleic acid encoding a mannosidase II; (ix) a nucleic acid encoding an α-1,3-glucosyltransferase; (x) a nucleic acid encoding a galactosyltransferase; and (xi) a nucleic acid encoding the α and β subunits of a glucosidase Such cells further can include a nucleic acid encoding a target protein as described herein.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, Genbank® Accession Nos, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description, and from the claims.

### DESCRIPTION OF DRAWINGS

FIG. 1A is a depiction of the nucleotide sequence of a light chain expression construct (SEQ ID NO:1) and a heavy chain expression construct (SEQ ID NO:2).

FIG. 1B is a depiction of the amino acid sequence of pYPS1 (SEQ ID NO:3) and pYPS2 protein (SEQ ID NO:4).

FIG. 1C is a depiction of the nucleotide sequence (SEQ ID NO:5) encoding the light chain (LC) of the anti-HER2 antibody, and a depiction of the amino acid sequence of the LC (SEQ ID NO:6), with the LIP2 prepro leader sequence underlined (LIP2 prepro leader sequence), the V_{L} domain sequence underlined with two lines (V_{L} domain); and the CK domain underlined with a dashed line (Ck1 domain).

FIG. 1D is a depiction of the nucleotide sequence (SEQ ID NO:7) encoding the heavy chain (HC) of the anti-HER2 antibody, and a depiction of the amino acid sequence of the HC (SEQ ID NO:8), with the LIP2 prepro leader sequence underlined (LIP2 prepro leader sequence), the V_{H} domain sequence underlined with two lines (V_{H} domain; the CH domain underlined with a dashed line (CH domain); and the yapsin cleavage site marked with a “I”.

FIG. 2 is a schematic of the genealogy of the strain constructed for single targeted copy integrations of the alphaHER2 heavy and light chains.

FIG. 3 is a photograph of a western blot of anti-HER2 antibody expressed in Yarrowia lipolytica strain Pold. The light and heavy chains were detected separately. Light chain was present at the correct molecular weight of 25 kDa but showed a tendency to dimerize. Heavy chain also was detected at the correct molecular weight of 50 kDa, but the majority was present as a degraded product with a molecular weight of approximately 32 kDa.

FIG. 4 is a schematic of a construct for disruption of YPS genes.

FIG. 5 is a photograph of two western blots of the heavy chain obtained from the culture supernatant of single yapsin deleted strains. In the upper panel, heavy chain was detected at two time points (48 h and 96 h) for the Δyps2 deletion, Δyps3 deletion, Δyps5 deletion, Δyps7 deletion, and Δypsx deletion strains, and the control strain (ctrl, yapsin non-deleted). In the lower panel, heavy chain was detected at the 96 h time point for two clones each of the Δyps1 deletion and Δyps4 deletion strains and the control strain.

FIG. 6 is a photograph of a western blot of the heavy chain obtained from the culture supernatants of a Δyps1 deletion strain, an URA-auxotrophic Δyps1 deletion strain, a Δyps1Δyps2 double deletion strain, a Δyps1Δyps3 double deletion strain, a Δyps1Δyps4 double deletion strain, and control strain (yapsin non-deleted).

FIG. 7 is a photograph of a silver stained sodium dodecyl sulfate polyacrylamide gel electrophoresis (SDS-PAGE) gel of the recombinant anti-HER2 antibody expressed in the Δyps1 Δyps2 and wild type (ctrl) strains. Reducing (left side) and non-reducing (right side) conditions are shown. Heavy chain derived degradation products are marked with an asterisk. Under non-reducing conditions, heavy chain proteolytic products were present both as a monomer and dimer in the control strain. Under reducing conditions, both glycosylated and unglycosylated versions of the heavy chain were observed. H2L2: fully assembled Ab; HC: heavy chain; LC: light chain.

### DETAILED DESCRIPTION

In general, this document provides methods and materials for reducing degradation of recombinant proteins in fungal cells such as Yarrowia (e.g., Y. lipolytica) or other related species of dimorphic yeast using genetically engineered cells that have deficiencies in two different yapsin peptidases, YPS1 protein (pYPS1) and YPS2 protein (pYPS2). Yapsins are glycophosphatidylinositol (GPI)-linked aspartic endopeptidases that have restricted substrate specificity and are localized on the cell surface. Yapsins can cleave C-terminally to paired basic residues (e.g., lysine-arginine and arginine-arginine); C-terminally to monobasic sites, with no preference of arginine over lysine; and between basic residues. See, e.g., Gagnon-Arsenault, et al., FEMS Yeast Res 6: 966-978 (2006).

The genetically engineered cells described herein can be used to produce recombinant target proteins. In some embodiments, the recombinant target proteins are capable of being trafficked through one or more steps of the Yarrowia lipolytica (or other related species of dimorphic yeast) secretory pathway, resulting in their N-glycosylation by the host cell machinery.

Suitable target proteins that can be recombinantly produced include pathogen proteins, lysosomal proteins (e.g., glucocerebrosidase, cerebrosidase, or galactocerebrosidase), insulin, glucagon, growth factors, cytokines, chemokines, a protein capable of binding to an Fc receptor, antibodies or fragments thereof, or fusions of any of the proteins to antibodies or fragments of antibodies (e.g., protein-Fc). Non-limiting examples of pathogen proteins include tetanus toxoid; diphtheria toxoid; and viral surface proteins (e.g., cytomegalovirus (CMV) glycoproteins B, H and gCIII; human immunodeficiency virus 1 (HIV-1) envelope glycoproteins; Rous sarcoma virus (RSV) envelope glycoproteins; herpes simplex virus (HSV) envelope glycoproteins; Epstein Barr virus (EBV) envelope glycoproteins; varicella-zoster virus (VZV) envelope glycoproteins; human papilloma virus (HPV) envelope glycoproteins; Influenza virus glycoproteins; and Hepatitis family surface antigens). Growth factors include, e.g., vascular endothelial growth factor (VEGF), Insulin-like growth factor (IGF), bone morphogenic protein (BMP), Granulocyte-colony stimulating factor (G-CSF), Granulocyte-macrophage colony stimulating factor (GM-CSF), Nerve growth factor (NGF); a Neurotrophin, Platelet-derived growth factor (PDGF), Erythropoietin (EPO), Thrombopoietin (TPO), Myostatin (GDF-8), Growth Differentiation factor-9 (GDF9), basic fibroblast growth factor (bFGF or FGF2), Epidermal growth factor (EGF), Hepatocyte growth factor (HGF). Cytokines include interleukins (e.g., IL-1 to IL-33 such as IL-1, IL-2, IL-3, IL-4, IL-5, IL-6, IL-7, IL-8, IL-9, IL-10, IL-12, IL-13, or IL-15) and interferons (e.g., interferon β or interferon γ). Chemokines include, e.g., I-309, TCA-3, MCP-1, MIP-1α, MIP-1(3, RANTES, C10, MRP-2, MARC, MCP-3, MCP-2, MRP-2, CCF18, MIP-1γ, Eotaxin, MCP-5, MCP-4, NCC-1, Ckβ10, HCC-1, Leukotactin-1, LEC, NCC-4, TARC, PARC, or Eotaxin-2. Also included are tumor glycoproteins (e.g., tumor-associated antigens), for example, carcinoembryonic antigen (CEA), human mucins, HER-2/neu, and prostate-specific antigen (PSA) [Henderson and Finn, Adv in Immunology, 62, pp. 217-56 (1996)].

In some embodiments, the target protein is associated with a lysosomal storage disorder (LSD). Non-limiting examples of target proteins that are associated with a LSD include, e.g., alpha-L-iduronidase, beta-D-galactosidase, beta-glucosidase, beta-hexosaminidase, beta-D-mannosidase, alpha-L-fucosidase, arylsulfatase B, arylsulfatase A, alpha-N-acetylgalactosaminidase, aspartylglucosaminidase, iduronate-2-sulfatase, alpha-glucosaminide-N-acetyltransferase, beta-D-glucoronidase, hyaluronidase, alpha-L-mannosidase, alpha-neuraminidase, phosphotransferase, acid lipase, acid ceramidase, sphingomyelinase, thioesterase, cathepsin K, and lipoprotein lipase.

In some embodiments, the target protein is an antibody. While the antibody can be any antibody, non-limiting examples of antibodies include an antibody that binds CD3 such as OKT3, Teplizumab, or Otelixizumab; an antibody that binds tumor necrosis factor (TNF) such as Adalimumab (Humira®) or Infliximab (Remicade®); an antibody that binds TNF receptor such as Etanercept (Enbrel®); an antibody that binds CD20 such as Ibritumomab tiuxetan (Zevalin®) or Rituximab (Mabthera®); an antibody that binds glycoprotein IIa/IIb receptor (GPIIa/IIb-R) such as Abeiximab (Reopro®); an antibody that binds IL2-receptor such as Basiliximab (Simulect®) or Daclizumab (Zenapax®), an antibody that binds to epidermal growth factor receptor (EGFR) such as Cetuximab (Erbitux®); an antibody that binds CD52 such as Alemtuzamab (Campath®); an antibody that binds CD 11a such as Efalizumab (Raptiva®); an antibody that binds vascular endothelial growth factor (VEGF) such as Bevacizumab (Avastin®), or an antibody that binds HER2 such as Trastuzamab (Herceptin®).

Target proteins also can be fusion proteins. Fusions proteins include, e.g., a fusion of (i) any protein described herein or fragment thereof with (ii) an antibody or fragment thereof. They also can be fusions of (i) and any of a variety of heterologous proteins, e.g., signal sequences derived from unrelated proteins, immunoglobulin heavy chain constant regions or parts of such regions, tag amino acid sequences (e.g., fluorescent proteins such as green fluorescent protein or variants of it), or sequences useful for affinity purification (e.g., poly-histidine such as hexahistidine, FLAG tag, or elastin-like polypeptide (ELP)).

Also of interest are antibody fragments (including antigen-binding antibody fragments). Such fragments can of any of the antibodies disclosed in this document. As used herein, the term “antibody fragment” refers to (a) an antigen-binding fragment or (b) an Fc part of the antibody that can interact with an Fc receptor. An antigen binding fragment can be, for example, a Fab, F(ab′)₂, Fv, and single chain Fv (scFv) fragment. An scFv fragment is a single polypeptide chain that includes both the heavy and light chain variable regions of the antibody from which the scFv is derived. In addition, diabodies [Poljak (1994) Structure 2(12):1121-1123; Hudson et al. (1999) J. Immunol. Methods 23(1-2):177-189] and intrabodies [Huston et al. (2001) Hum. Antibodies 10(3-4):127-142; Wheeler et al. (2003) Mol. Ther. 8(3):355-366; Stocks (2004) Drug Discov. Today 9(22): 960-966] are examples of recombinant proteins that can be produced.

Target proteins can be encoded by one or more (e.g., two, three, four, or five) nucleic acids, optionally in one or more (e.g., two, three, four, or five) expression vectors, encoding one or more polypeptide chains of the target protein. Thus, for example, both chains (e.g., light and heavy chains or a fragment of one or both) of an antibody or an antigen-binding fragment of an antibody can be expressed by a single open reading frame (ORF) in a single expression vector or by two ORFs, either in a single expression vector or two separate expression vectors. Thus, an antibody scFV containing the light and heavy chain variable regions of an antibody would generally be encoded by a single ORF. On the other hand, the light and heavy chains a whole IgG antibody, a Fab fragment, or a F(ab′)2 fragment would most commonly (but not necessarily) be expressed by separate ORFs within two separate nucleic acids, each generally (but again not necessarily) in a separate expression vector. The same principles described above for antibodies and antigen-binding fragments of antibodies are understood to apply to other proteins composed of one or more (e.g., two, three, four, or five) non-identical polypeptide chains.

Target proteins also can be joined to one or more of a polymer, a carrier, an adjuvant, an immunotoxin, or a detectable (e.g., fluorescent, luminescent, or radioactive) moiety. For example, a recombinant protein can be joined to polyethyleneglycol, which can be used to increase the molecular weight of small proteins and/or increase circulation residence time.

### Genetically Engineered Cells

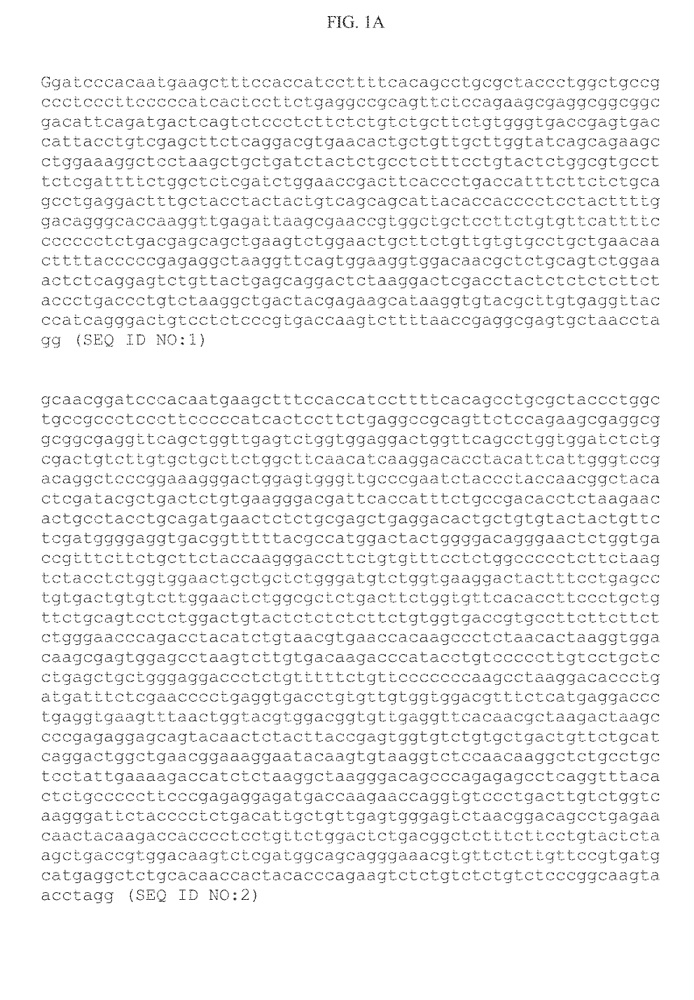

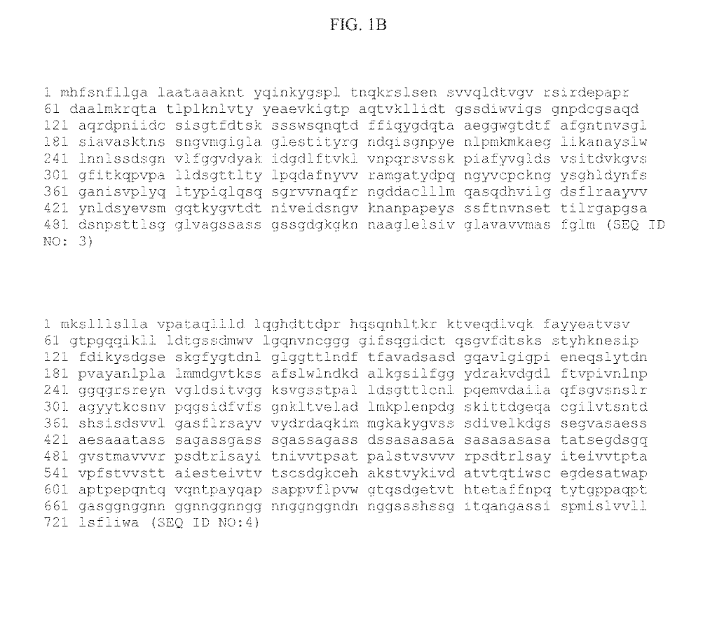

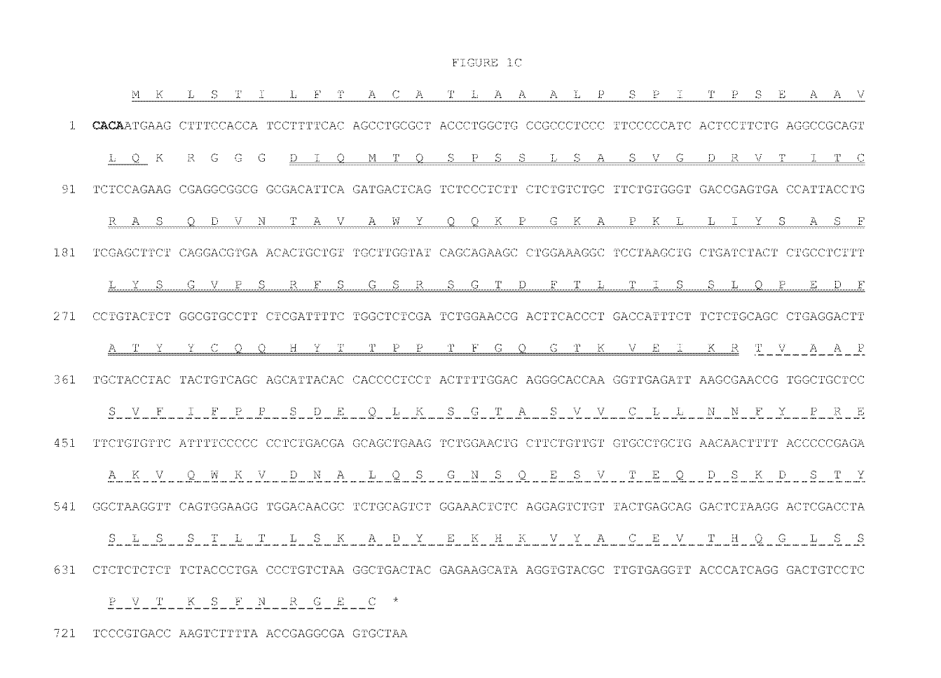

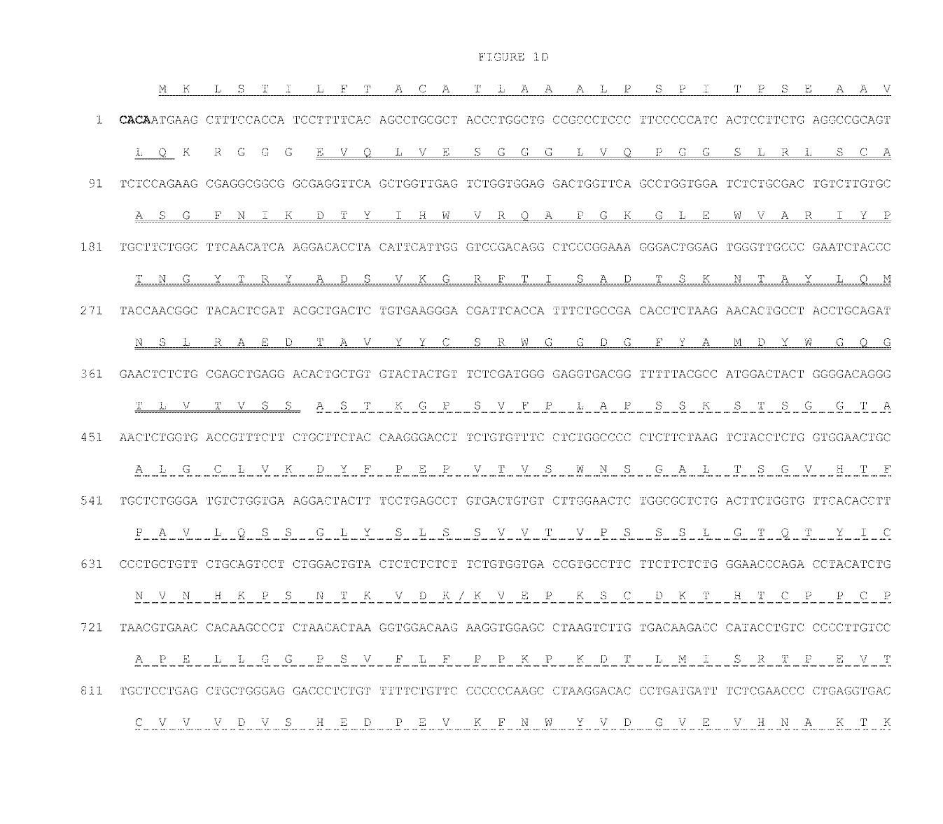

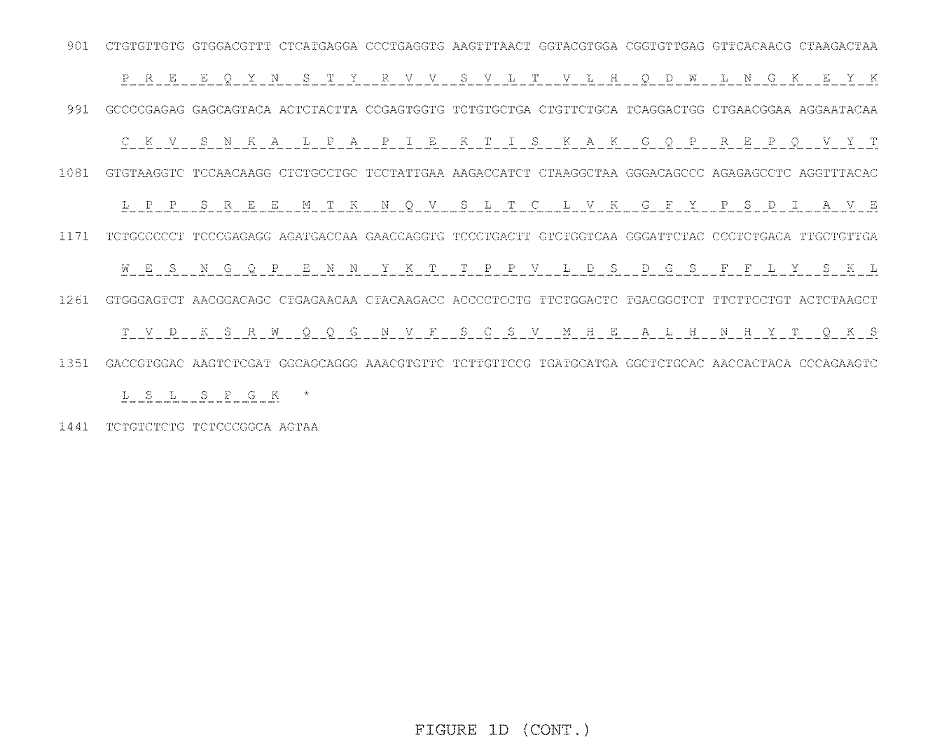

Genetically engineered cells described herein (e.g., Yarrowia cells) contain deficiencies in pYPS1 and pYPS2 activities. For example, such a genetically engineered cell may not produce detectable levels of a functional pYPS1 and/or a functional pYPS2. Such deficiencies can be produced in Yarrowia cells by, for example, deleting or disrupting at least two endogenous yapsin genes, e.g., YPS1 (Genolevures Ref No. YALI0E10175g; Gene ID: 2912589) and YPS2 (Genolevures Ref No. YALI0E22374g; Gene ID: 2912981), which encode pYPS1 and pYPS2, respectively. The amino acid sequence of pYPS1 and pYPS2 are set forth in SEQ ID NO:3 and SEQ ID NO:4, respectively (see FIG. 1B). See also GenBank Accession No. XP_{—}503768.1, GI:50552716 and XP_{—}504265.1, GI:50553708, respectively.

Homologous recombination can be used to disrupt an endogenous gene. For example, a “gene replacement” vector can be constructed in such a way to include a selectable marker gene. The selectable marker gene can be operably linked, at both 5′ and 3′ end, to portions of the gene of sufficient length to mediate homologous recombination. The selectable marker can be one of any number of genes which either complement host cell auxotrophy or provide antibiotic resistance, including URA3, LEU2 and HIS3 genes. Other suitable selectable markers include the CAT gene, which confers chloramphenicol resistance to yeast cells, or the lacZ gene, which results in blue colonies due to the expression of β-galactosidase. Linearized DNA fragments of the gene replacement vector then are introduced into the cells using methods well known in the art (see below). Integration of the linear fragments into the genome and the disruption of the gene can be determined based on the selection marker and can be verified by, for example, Southern blot analysis. In some embodiments, disruption of the gene results in the genetically engineered strain not producing detectable levels of mRNA molecules encoding a functional pYPS1 and a functional pYPS2.

Subsequent to its use in selection, a selectable marker can be removed from the genome of the host cell by, e.g., Cre-loxP systems (see, e.g., Gossen et al. (2002) Ann. Rev. Genetics 36:153-173 and U.S. Application Publication No. 20060014264). The process of marker removal is referred to as “curing.”

Alternatively, a gene replacement vector can be constructed in such a way as to include a portion of the gene to be disrupted, where the portion is devoid of any endogenous gene promoter sequence and encodes none, or an inactive fragment of, the coding sequence of the gene. An “inactive fragment” is a fragment of the gene that encodes a protein having, e.g., less than about 10% (e.g., less than about 9%, less than about 8%, less than about 7%, less than about 6%, less than about 5%, less than about 4%, less than about 3%, less than about 2%, less than about 1%, or 0%) of the activity of the protein produced from the full-length coding sequence of the gene. Such a portion of the gene is inserted in a vector in such a way that no known promoter sequence is operably linked to the gene sequence, but that a stop codon and a transcription termination sequence are operably linked to the portion of the gene sequence. This vector can be subsequently linearized in the portion of the gene sequence and transformed into a cell. By way of single homologous recombination, this linearized vector is then integrated in the endogenous counterpart of the gene.

In some embodiments, an RNA molecule can be introduced or expressed that interferes with the functional expression of a protein having pYPS1 and/or pYPS2 activity. RNA molecules include, e.g., small-interfering RNA (siRNA), short hairpin RNA (shRNA), anti-sense RNA, or micro RNA (miRNA).

In some embodiments, the promoter or enhancer elements of one or more endogenous genes encoding a protein having pYPS1 and/or pYPS2 activity can be altered such that the expression of their encoded proteins is altered.

Cells suitable for genetic engineering include Yarrowia cells such as Y. lipolytica cells and other related dimorphic yeast cells. Such cells, prior to the genetic engineering as specified herein, can be obtained from a variety of commercial sources and research resource facilities, such as, for example, the American Type Culture Collection (ATCC) (Manassas, Va.). In one embodiment, the pold strain of Y. lipolytica is used. The pold strain is available at the Centre International de Ressources Microbienne, CLIB culture collection under the accession number 139. In the pold strain, the secreted alkaline extracellular protease AEP (gene XPR2) has been deleted and the acid extracellular protease AXP1 (gene AXP) can either be deleted by gene disruption and insertion of a target gene or controlled by pH of the fermentation medium.

Genetically engineered cells described herein further can include deficiencies in other aspartic proteases, e.g., aspartic proteases classified under EC 3.4.23 such as proteinase A (encoded by PEP4 gene).

Genetically engineered cells described herein further can include a nucleic acid encoding a target protein (e.g., a target protein described above such as an antibody). The terms “nucleic acid” and “polynucleotide” are used interchangeably herein, and refer to both RNA and DNA, including cDNA, genomic DNA, synthetic DNA, and DNA (or RNA) containing nucleic acid analogs. Nucleic acids can have any three-dimensional structure. A nucleic acid can be double-stranded or single-stranded (i.e., a sense strand or an antisense strand). Non-limiting examples of nucleic acids include genes, gene fragments, exons, introns, messenger RNA (mRNA), transfer RNA, ribosomal RNA, siRNA, micro-RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs. “Polypeptide” and “protein” are used interchangeably herein and mean any peptide-linked chain of amino acids, regardless of length or post-translational modification.

An “isolated nucleic acid” refers to a nucleic acid that is separated from other nucleic acid molecules that are present in a naturally-occurring genome, including nucleic acids that normally flank one or both sides of the nucleic acid in a naturally-occurring genome (e.g., a yeast genome). The term “isolated” as used herein with respect to nucleic acids also includes any non-naturally-occurring nucleic acid sequence, since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences as well as DNA that is incorporated into a vector, an autonomously replicating plasmid, a virus (e.g., any paramyxovirus, retrovirus, lentivirus, adenovirus, or herpes virus), or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel (e.g., electrophoretic gel) slices containing a genomic DNA restriction digest, is not considered an isolated nucleic acid.

The term “exogenous” as used herein with reference to nucleic acid and a particular host cell refers to any nucleic acid that does not occur in (and cannot be obtained from) that particular cell as found in nature. Thus, a non-naturally-occurring nucleic acid is considered to be exogenous to a host cell once introduced into the host cell. It is important to note that non-naturally-occurring nucleic acids can contain nucleic acid subsequences or fragments of nucleic acid sequences that are found in nature provided that the nucleic acid as a whole does not exist in nature. For example, a nucleic acid molecule containing a genomic DNA sequence within an expression vector is non-naturally-occurring nucleic acid, and thus is exogenous to a host cell once introduced into the host cell, since that nucleic acid molecule as a whole (genomic DNA plus vector DNA) does not exist in nature. Thus, any vector, autonomously replicating plasmid, or virus (e.g., retrovirus, adenovirus, or herpes virus) that as a whole does not exist in nature is considered to be non-naturally-occurring nucleic acid. It follows that genomic DNA fragments produced by PCR or restriction endonuclease treatment as well as cDNAs are considered to be non-naturally-occurring nucleic acid since they exist as separate molecules not found in nature. It also follows that any nucleic acid containing a promoter sequence and polypeptide-encoding sequence (e.g., cDNA or genomic DNA) in an arrangement not found in nature is non-naturally-occurring nucleic acid. A nucleic acid that is naturally-occurring can be exogenous to a particular cell. For example, an entire chromosome isolated from a cell of yeast x is an exogenous nucleic acid with respect to a cell of yeast y once that chromosome is introduced into a cell of yeast y.

A recombinant nucleic acid can be in introduced into the cell in the form of an expression vector such as a plasmid, phage, transposon, cosmid or virus particle using a variety of methods such as the spheroplast technique or the whole-cell lithium chloride yeast transformation method. Other methods useful for transformation of plasmids or linear nucleic acid vectors into cells are described in, for example, U.S. Pat. No. 4,929,555; Hinnen et al. (1978) Proc. Nat. Acad. Sci. USA 75:1929; Ito et al. (1983) J. Bacteriol. 153:163; U.S. Pat. No. 4,879,231; and Sreekrishna et al. (1987) Gene 59:115. Electroporation and PEG1000 whole cell transformation procedures may also be used, as described by Cregg and Russel, Methods in Molecular Biology: Pichia Protocols, Chapter 3, Humana Press, Totowa, N.J., pp. 27-39 (1998).

Transformed yeast cells can be selected using techniques including, but not limited to, culturing auxotrophic cells after transformation in the absence of the biochemical product required (due to the cell's auxotrophy), selection for and detection of a new phenotype, or culturing in the presence of an antibiotic which is toxic to the yeast in the absence of a resistance gene contained in the transformants. Transformants can also be selected and/or verified by integration of the expression cassette into the genome, which can be assessed by, e.g., Southern blot or PCR analysis.

Prior to introducing the vectors into a cell such as a Yarrowia cell, the vectors can be grown (e.g., amplified) in bacterial cells such as Escherichia coli (E. coli). The vector DNA can be isolated from bacterial cells by any of the methods known in the art which result in the purification of vector DNA from the bacterial milieu. The purified vector DNA can be extracted extensively with phenol, chloroform, and ether, to ensure that no E. coli proteins are present in the plasmid DNA preparation.

Integrative vectors are disclosed, e.g., in U.S. Pat. No. 4,882,279. Integrative vectors generally include a serially arranged sequence of at least a first insertable DNA fragment, a selectable marker gene, and a second insertable DNA fragment. The first and second insertable DNA fragments are each about 200 (e.g., about 250, about 300, about 350, about 400, about 450, about 500, or about 1000 or more) nucleotides in length and have nucleotide sequences which are homologous to portions of the genomic DNA of the species to be transformed. A nucleotide sequence containing a gene of interest (e.g., a gene encoding a target protein) for expression is inserted in this vector between the first and second insertable DNA fragments whether before or after the marker gene. Integrative vectors can be linearized prior to yeast transformation to facilitate the integration of the nucleotide sequence of interest into the host cell genome.

An expression vector can feature a recombinant nucleic acid under the control of a yeast (e.g., Yarrowia lipolytica, Arxula adeninivorans, or other related dimorphic yeast species) promoter, which enables them to be expressed in yeast. Suitable yeast promoters include the TEF1, HP4D, GAP, POX2, ADC1, TPI1, ADH2, POX, and Gal10 promter. See, e.g., Madzak et al., (2000) J. Mol. Microbiol. Biotechnol. 2:207-216; Guarente et al. (1982) Proc. Natl. Acad. Sci. USA 79(23):7410. Additional suitable promoters are described in, e.g., Zhu and Zhang (1999) Bioinformatics 15(7-8):608-611 and U.S. Pat. No. 6,265,185.

A promoter can be constitutive or inducible (conditional). A constitutive promoter is understood to be a promoter whose expression is constant or substantially constant under the standard culturing conditions. Inducible promoters are promoters that are responsive to one or more induction cues. For example, an inducible promoter can be chemically regulated (e.g., a promoter whose transcriptional activity is regulated by the presence or absence of a chemical inducing agent such as an alcohol, tetracycline, a steroid, a metal, or other small molecule) or physically regulated (e.g., a promoter whose transcriptional activity is regulated by the presence or absence of a physical inducer such as light or high or low temperatures). An inducible promoter can also be indirectly regulated by one or more transcription factors that are themselves directly regulated by chemical or physical cues.

Genetically engineered cells described herein further can include one or more additional modifications such that the cell produces the desired N-glycan on the target protein. The additional modifications can include one or more of (i) deletion or disruption of an endogenous gene encoding a protein having N-glycosylation activity; (ii) introduction of a recombinant nucleic acid encoding a mutant form of a protein (e.g., endogenous or exogenous protein) having N-glycosylation activity (i.e., expressing a mutant protein having an N-glycosylation activity); (iii) introduction or expression of an RNA molecule that interferes with the functional expression of a protein having the N-glycosylation activity; (iv) introduction of a recombinant nucleic acid encoding a wild-type (e.g., endogenous or exogenous) protein having N-glycosylation activity (i.e., expressing a protein having an N-glycosylation activity); and (v) altering the promoter or enhancer elements of one or more endogenous genes encoding proteins having N-glycosylation activity to thus alter the expression of their encoded proteins. It is understood that item (ii) includes, e.g., replacement of an endogenous gene with a gene encoding a protein having greater N-glycosylation activity relative to the endogenous gene so replaced. Genetic engineering also includes altering an endogenous gene encoding a protein having an N-glycosylation activity to produce a protein having additions (e.g., a heterologous sequence), deletions, or substitutions (e.g., mutations such as point mutations; conservative or non-conservative mutations). Mutations can be introduced specifically (e.g., site-directed mutagenesis or homologous recombination) or can be introduced randomly (for example, cells can be chemically mutagenized as described in, e.g., Newman and Ferro-Novick (1987) J. Cell Biol. 105(4):1587. Modifications can include, for example, those described in WO 2011/061629 and WO 2011/039634.

Such additional genetic modifications can result in one or more of (i) an increase in one or more N-glycosylation activities in the genetically modified cell, (ii) a decrease in one or more N-glycosylation activities in the genetically modified cell, (iii) a change in the localization or intracellular distribution of one or more N-glycosylation activities in the genetically modified cell, or (iv) a change in the ratio of one or more N-glycosylation activities in the genetically modified cell. It is understood that an increase in the amount of an N-glycosylation activity can be due to overexpression of one or more proteins having N-glycosylation activity, an increase in copy number of an endogenous gene (e.g., gene duplication), or an alteration in the promoter or enhancer of an endogenous gene that stimulates an increase in expression of the protein encoded by the gene. A decrease in one or more N-glycosylation activities can be due to overexpression of a mutant form (e.g., a dominant negative form) of one or more proteins having N-glycosylation altering activities, introduction or expression of one or more interfering RNA molecules that reduce the expression of one or more proteins having an N-glycosylation activity, or deletion or disruption of one or more endogenous genes that encode a protein having N-glycosylation activity.

It is understood that genetically engineered modifications can be conditional. For example, a gene can be conditionally deleted using, e.g., a site-specific DNA recombinase such as the Cre-loxP system (see, e.g., Gossen et al. (2002) Ann. Rev. Genetics 36:153-173 and U.S. Application Publication No. 20060014264).

Proteins having N-glycosylation activity include, for example, an Outer CHain elongation (OCH1) protein, an α-1,2-mannosidase, an Asparagine Linked Glycosylation 3 (ALG3) protein, an α-1,3-glucosyltransferase, a glucosidase, a mannosidase II, a GlcNAc-transferase I (GnT I), a GlcNAc-transferase II (GnT II), or a galactosyltransferase (Gal T).

A desired N-glycan on a secreted protein can be based, for example, on either a Man₅GlcNAc₂ or Man₃GlcNAc₂ structure. For example, to produce a Man₅GlcNAc₂ base structure, Yarrowia cells can be engineered such that α-1,2-mannosidase activity is increased in an intracellular compartment and OCH1 activity is decreased. To produce a Man₃GlcNAc₂ base structure, activity of ALG3 and, in some embodiments, OCH1, is decreased, and activity of α-1,2-mannosidase and, in some embodiments, activity of α-1,3-glucosyltransferase, is increased. The N-glycan profile of proteins produced in such yeast cells can be altered by further engineering the cells to contain one or more of the following activities: GlcNAc transferase I (GnT I) activity, mannosidase II (Man II) activity, GlcNAc transferase II (GnT II) activity, glucosidase II activity, and galactosyltransferase (Gal T) activity. For example, expressing GnT I in a Yarrowia cell producing Man₅GlcNAc₂ or Man₃GlcNAc₂N-glycans results in the transfer of a GlcNAc moiety to the Man₅GlcNAc₂ or Man₃GlcNAc₂N-glycans such that GlcNAcMan₅GlcNAc₂ or GlcNAcMan₃GlcNAc₂ N-glycans, respectively, are produced. In cells producing GlcNAcMan₅GlcNAc₂ N-glycans, expressing a mannosidase II results in two mannose residues being removed from GlcNAcMan₅GlcNAc₂ N-glycans to produce GlcNAcMan₃GlcNAc₂ N-glycans. In cells producing GlcNAcMan₃GlcNAc₂ N-glycans, expressing GnT II results in the transfer of another GlcNAc moiety to GlcNAcMan₃GlcNAc₂ N-glycans to produce GlcNAc₂Man₃GlcNAc₂ N-glycans. Expressing Gal T in cells producing GlcNAcMan₃GlcNAc₂ or GlcNAc₂Man₃GlcNAc₂ N-glycans results in the transfer of galactose to GlcNAcMan₃GlcNAc₂ or GlcNAc₂Man₃GlcNAc₂ N-glycans to produce GalGlcNAcMan₃GlcNAc₂ or Gal₂GlcNAc₂Man₃GlcNAc₂ N-glycans. In some embodiments, a glucosidase (e.g., by expressing α and β subunits) can be expressed to increase production of the Man₃GlcNAc₂ base structure.

The genes encoding proteins having N-glycosylation activity can be from any species containing such genes. Exemplary fungal species from which genes encoding proteins having N-glycosylation activity can be obtained include, without limitation, Pichia anomala, Pichia bovis, Pichia canadensis, Pichia carsonii, Pichia farinose, Pichia fermentans, Pichia fluxuum, Pichia membranaefaciens, Pichia membranaefaciens, Candida valida, Candida albicans, Candida ascalaphidarum, Candida amphixiae, Candida Antarctica, Candida atlantica, Candida atmosphaerica, Candida blattae, Candida carpophila, Candida cerambycidarum, Candida chauliodes, Candida corydalis, Candida dosseyi, Candida dubliniensis, Candida ergatensis, Candida fructus, Candida glabrata, Candida fermentati, Candida guilliermondii, Candida haemulonii, Candida insectamens, Candida insectorum, Candida intermedia, Candida jeffresii, Candida keftr, Candida krusei, Candida lusitaniae, Candida lyxosophila, Candida maltosa, Candida membranifaciens, Candida milleri, Candida oleophila, Candida oregonensis, Candida parapsilosis, Candida quercitrusa, Candida shehatea, Candida temnochilae, Candida tenuis, Candida tropicalis, Candida tsuchiyae, Candida sinolaborantium, Candida sojae, Candida viswanathii, Candida utilis, Pichia membranaefaciens, Pichia silvestris, Pichia membranaefaciens, Pichia chodati, Pichia membranaefaciens, Pichia menbranaefaciens, Pichia minuscule, Pichia pastoris, Pichia pseudopolymorpha, Pichia quercuum, Pichia robertsii, Pichia saitoi, Pichia silvestrisi, Pichia strasburgensis, Pichia terricola, Pichia vanriji, Pseudozyma Antarctica, Rhodosporidium toruloides, Rhodotorula glutinis, Saccharomyces bayanus, Saccharomyces bayanus, Saccharomyces momdshuricus, Saccharomyces uvarum, Saccharomyces bayanus, Saccharomyces cerevisiae, Saccharomyces bisporus, Saccharomyces chevalieri, Saccharomyces delbrueckii, Saccharomyces exiguous, Saccharomyces fermentati, Saccharomyces fragilis, Saccharomyces marxianus, Saccharomyces mellis, Saccharomyces rosei, Saccharomyces rouxii, Saccharomyces uvarum, Saccharomyces willianus, Saccharomycodes ludwigii, Saccharomycopsis capsularis, Saccharomycopsis fibuligera, Saccharomycopsis fibuligera, Endomyces hordei, Endomycopsis fobuligera. Saturnispora saitoi, Schizosaccharomyces octosporus, Schizosaccharomyces pombe, Schwanniomyces occidentalis, Torulaspora delbrueckii, Torulaspora delbrueckii, Saccharomyces dairensis, Torulaspora delbrueckii, Torulaspora fermentati, Saccharomyces fermentati, Torulaspora delbrueckii, Torulaspora rosei, Saccharomyces rosei, Torulaspora delbrueckii, Saccharomyces rosei, Torulaspora delbrueckii, Saccharomyces delbrueckii, Torulaspora delbrueckii, Saccharomyces delbrueckii, Zygosaccharomyces mongolicus, Dorulaspora globosa, Debaryomyces globosus, Torulopsis globosa, Trichosporon cutaneum, Trigonopsis variabilis, Williopsis californica, Williopsis saturnus, Zygosaccharomyces bisporus, Zygosaccharomyces bisporus, Debaryomyces disporua. Saccharomyces bisporas, Zygosaccharomyces bisporus, Saccharomyces bisporus, Zygosaccharomyces mellis, Zygosaccharomyces priorianus, Zygosaccharomyces rouxiim, Zygosaccharomyces rouxii, Zygosaccharomyces barkeri, Saccharomyces rouxii, Zygosaccharomyces rouxii, Zygosaccharomyces major, Saccharomyces rousii, Pichia anomala, Pichia bovis, Pichia Canadensis, Pichia carsonii, Pichia farinose, Pichia fermentans, Pichia fiuxuum, Pichia membranaefaciens, Pichia pseudopolymorpha, Pichia quercuum, Pichia robertsii, Pseudozyma Antarctica, Rhodosporidium toruloides, Rhodosporidium toruloides, Rhodotorula glutinis, Saccharomyces bayanus, Saccharomyces bayanus, Saccharomyces bisporus, Saccharomyces cerevisiae, Saccharomyces chevalieri, Saccharomyces delbrueckii, Saccharomyces fermentati, Saccharomyces fragilis, Saccharomycodes ludwigii, Schizosaccharomyces pombe, Schwanniomyces occidentalis, Torulaspora delbrueckii, Torulaspora globosa, Trigonopsis variabilis, Williopsis californica, Williopsis saturnus, Zygosaccharomyces bisporus, Zygosaccharomyces mellis, Zygosaccharomyces rouxii, or any other fungi (e.g., yeast) known in the art or described herein.

Exemplary lower eukaryotes also include various species of Aspergillus including, but not limited to, Aspergillus caesiellus, Aspergillus candidus, Aspergillus carneus, Aspergillus clavatus, Aspergillus deflectus, Aspergillus flavus, Aspergillus fumigatus, Aspergillus glaucus, Aspergillus nidulans, Aspergillus niger, Aspergillus ochraceus, Aspergillus oryzae, Aspergillus parasiticus, Aspergillus penicilloides, Aspergillus restrictus, Aspergillus sojae, Aspergillus sydowi, Aspergillus tamari, Aspergillus terreus, Aspergillus ustus, or Aspergillus versicolor.

Exemplary protozoal genera from which genes encoding proteins having N-glycosylation activity can be obtained include, without limitation, Blastocrithidia, Crithidia, Endotrypanum, Herpetomonas, Leishmania, Leptomonas, Phytomonas, Trypanosoma (e.g., T. bruceii, T. gambiense, T. rhodesiense, and T. cruzi), and Wallaceina.

For example, the gene encoding GnT I can be obtained from human (Swiss Protein Accession No. P26572), rat, Arabidopsis, mouse, or Drosophila; the gene encoding GntII can be obtained from human, rat (Swiss Protein Accession No. Q09326), Arabidopsis, or mouse; the gene encoding Man II can be obtained from human, rat, Arabidopsis, mouse, Drosophila (Swiss Protein Accession No. Q24451); and the gene encoding GalT can be obtained from human (Swiss Protein Accession No. P15291), rat, mouse, or bovine.

In some embodiments, a genetically engineered cell described herein can include one or more of the following modifications in addition to having deficiencies in pYPS1 and pYPS2 activities. For example, a genetically engineered cell further can lack the OCH1 (GenBank Accession No: AJ563920) gene or gene product (mRNA or protein) thereof. In some embodiments, a genetically engineered cell further can lack the ALG3 (Genbank® Accession Nos: XM_{—}503488, Genolevures Ref: YALI0E03190g) gene or gene product (mRNA or protein) thereof. In some embodiments, a genetically engineered cell further expresses (e.g., overexpresses) an α-1,3-glucosyltransferase (e.g., ALG6, Genbank® Accession Nos: XM_{—}502922, Genolevures Ref: YALI0D17028g) protein. In some embodiments, a genetically engineered cell further expresses an α-1,2-mannosidase (e.g., Genbank Accession No.: AF212153) protein. In some embodiments, a genetically engineered cell further expresses a GlcNAc-transferase I (e.g., Swiss Prot. Accession No. P26572) protein. In some embodiments, a genetically engineered cell further expresses a mannosidase II protein or catalytic domain thereof (e.g., Swiss Prot. Accession No. Q24451). In some embodiments, a genetically engineered cell further expresses a galactosyltransferase I protein or catalytic domain thereof (e.g., Swiss Prot. Accession No. P15291). In some embodiments, the genetically engineered cell further expresses a GlcNAc-transferase II protein or catalytic domain thereof (e.g., Swiss Prot. Accession No. Q09326). In some embodiments, the genetically engineered cell further expresses an alpha or beta subunit (or both the alpha and the beta subunit) of a glucosidase II such as the glucosidase II of Yarrowia lipolytica, Trypanosoma brucei or Aspergillus niger. A genetically engineered cell can have any combination of these modifications.

For example, in some embodiments, a genetically engineered cell can lack the OCH1 gene and express an α-1,2-mannosidase, GlcNAc-transferase I, mannosidase II, and a galactosyltransferase I. In some embodiment, a genetically engineered cell can lack the ALG3 gene, and express an α-1,2-mannosidase, GlcNAc-transferase I, GlcNAc-transferase I, and a galactosyltransferase I. Such a genetically engineered cell further can express an α-1,3-glucosyltransferase and/or express alpha and beta subunits of a glucosidase II and/or lack the OCH1 gene.

One of more of such proteins can be fusion proteins that contain a heterologous targeting sequence. For example, the α-1,2-mannosidase can have an HDEL endoplasmic reticulum (ER)-retention amino acid sequence. It is understood that any protein having N-glycosylation activity can be engineered into a fusion protein comprising an HDEL sequence. Other proteins can have heterologous sequences that target the protein to the Golgi apparatus. For example, the first 100 N-terminal amino acids encoded by the yeast Kre2p gene, the first 36 N-terminal amino acids (Swiss Prot. Accession No. P38069) encoded by the S. cerevisiae Mnn2 gene, or the first 46 N-terminal amino acids encoded by the S. cerevisiae Mnn2p gene can be used to target proteins to the Golgi. As such, nucleic acids encoding a protein to be expressed in a fungal cell can include a nucleotide sequence encoding a targeting sequence to target the encoded protein to an intracellular compartment. For example, the α-1,2-mannosidase can be targeted to the ER, while the GnT I, GnT II, mannosidase, and Gal T can be targeted to the Golgi.

In embodiments where a target protein or protein having N-glycosylation activity is derived from a cell that is of a different type (e.g., of a different species) than the cell into which the protein is to be expressed, a nucleic acid encoding the protein can be codon-optimized for expression in the particular cell of interest. For example, a nucleic acid encoding a protein having N-glycosylation from Trypanosoma brucei can be codon-optimized for expression in a yeast cell such as Y. lipolytica. Such codon-optimization can be useful for increasing expression of the protein in the cell of interest. Methods for codon-optimizing a nucleic acid encoding a protein are known in the art and described in, e.g., Gao et al. (Biotechnol. Prog. (2004) 20(2): 443-448), Kotula et al. (Nat. Biotechn. (1991) 9, 1386-1389), and Bennetzen et al. (J. Biol. Chem. (1982) 257(6):2036-3031). Table 1 shows the codon usage for Yarrowia lipolytica. Data was derived from 2,945,919 codons present in 5,967 coding sequences. The contents of Table 1 were obtained from a Codon Usage Database, which can be found at world wide web at kazusa.or.jp/codon/cgi-bin/showcodon.cgi?species=284591.

TABLE 1
Yarrowia lipolytica Codon Usage Table

| | | | |
|---|---|---|---|
| UUU 15.9(46804) | CU 21.8(64161) | AU 6.8(20043) | GU 6.1(17849) |
| UUC 23.0(67672) | CC 20.6(60695) | AC 23.1(68146) | GC 6.1(17903) |
| UUA 1.8(5280) | CA 7.8(22845) | AA 0.8(2494) | GA 0.4(1148) |
| UUG 10.4(30576) | CG 15.4(45255) | AG 0.8(2325) | GG 12.1(35555) |
| CUU 13.2(38890) | CU 17.4(51329) | AU 9.6(28191) | GU 6.0(17622) |
| CUC 22.6(66461) | CC 23.3(68633) | AC 14.4(42490) | GC 4.4(12915) |
| CUA 5.3(15548) | CA 6.9(20234) | AA 9.8(28769) | GA 21.7(63881) |
| CUG 33.5(98823) | CG 6.8(20042) | AG 32.1(94609) | GG 7.7(22606) |
| AUU 22.4(66134) | CU 16.2(47842) | AU 8.9(26184) | GU 6.7(19861) |
| AUC 24.4(71810) | CC 25.6(75551) | AC 31.3(92161) | GC 9.8(28855) |
| AUA 2.2(6342) | CA 10.5(30844) | AA 12.4(36672) | GA 8.4(24674) |
| AUG 22.6(66620) | CG 8.5(25021) | AG 46.5(136914) | GG 2.4(7208) |
| GUU 15.8(46530) | CU 25.5(75193) | AU 21.5(63259) | GU 16.6(48902) |
| GUC 21.5(63401) | CC 32.7(96219) | AC 38.3(112759) | GC 21.8(64272) |
| GUA 4.0(11840) | CA 11.2(32999) | AA 18.8(55382) | GA 20.9(61597) |
| GUG 25.7(75765) | CG 8.9(26190) | AG 46.2(136241) | GG 4.4(12883) |

Tablefields are shown as [triplet] [frequency: per thousand] ([number]).

In some embodiments, human target proteins can be introduced into the cell and one or more endogenous yeast proteins having N-glycosylation activity can be suppressed (e.g., deleted or mutated). Techniques for “humanizing” a fungal glycosylation pathway are described in, e.g., Choi et al. (2003) Proc. Natl. Acad. Sci. USA 100(9):5022-5027; Vervecken et al. (2004) Appl. Environ. Microb. 70(5):2639-2646; and Gerngross (2004) Nature Biotech. 22(11):1410-1414.

Where the genetic engineering involves, e.g., changes in the expression of a protein or expression of an exogenous protein (including a mutant form of an endogenous protein), a variety of techniques can be used to determine if the genetically engineered cells express the protein. For example, the presence of mRNA encoding the protein or the protein itself can be detected using, e.g., Northern Blot or RT-PCR analysis or Western Blot analysis, respectively. The intracellular localization of a protein having N-glycosylation activity can be analyzed by using a variety of techniques, including subcellular fractionation and immunofluorescence.

Methods for detecting glycosylation of a target protein include DNA sequencer-assisted (DSA), fluorophore-assisted carbohydrate electrophoresis (FACE) or surface-enhanced laser desorption/ionization time-of-flight mass spectrometry (SELDI-TOF MS). For example, an analysis can utilize DSA-FACE in which, for example, glycoproteins are denatured followed by immobilization on, e.g., a membrane. The glycoproteins can then be reduced with a suitable reducing agent such as dithiothreitol (DTT) or β-mercaptoethanol. The sulfhydryl groups of the proteins can be carboxylated using an acid such as iodoacetic acid. Next, the N-glycans can be released from the protein using an enzyme such as N-glycosidase F. N-glycans, optionally, can be reconstituted and derivatized by reductive amination. The derivatized N-glycans can then be concentrated. Instrumentation suitable for N-glycan analysis includes, e.g., the ABI PRISM® 377 DNA sequencer (Applied Biosystems). Data analysis can be performed using, e.g., GENESCAN® 3.1 software (Applied Biosystems). Optionally, isolated mannoproteins can be further treated with one or more enzymes to confirm their N-glycan status. Additional methods of N-glycan analysis include, e.g., mass spectrometry (e.g., MALDI-TOF-MS), high-pressure liquid chromatography (HPLC) on normal phase, reversed phase and ion exchange chromatography (e.g., with pulsed amperometric detection when glycans are not labeled and with UV absorbance or fluorescence if glycans are appropriately labeled). See also Callewaert et al. (2001) Glycobiology 11(4):275-281 and Freire et al. (2006) Bioconjug. Chem. 17(2):559-564.

Where any of the genetic modifications of the genetically engineered cells described herein are inducible or conditional on the presence of an inducing cue (e.g., a chemical or physical cue), the genetically engineered cell can, optionally, be cultured in the presence of an inducing agent before, during, or subsequent to the introduction of the nucleic acid. For example, following introduction of the nucleic acid encoding a target protein, the cell can be exposed to a chemical inducing agent that is capable of promoting the expression of one or more proteins having N-glycosylation activity. Where multiple inducing cues induce conditional expression of one or more proteins having N-glycosylation activity, a cell can be contacted with multiple inducing agents.

Target proteins modified to include the desired N-glycan can be isolated from the genetically engineered cell. The modified target protein can be maintained within the yeast cell and released upon cell lysis or the modified target protein can be secreted into the culture medium via a mechanism provided by a coding sequence (either native to the exogenous nucleic acid or engineered into the expression vector), which directs secretion of the protein from the cell. The presence of the modified target protein in the cell lysate or culture medium can be verified by a variety of standard protocols for detecting the presence of the protein, Such protocols can include, but are not limited to, immunoblotting or radioimmunoprecipitation with an antibody specific for the altered target protein (or the target protein itself), binding of a ligand specific for the altered target protein (or the target protein itself), or testing for a specific enzyme activity of the modified target protein (or the target protein itself).

In some embodiments, at least about 25% of the target proteins isolated from the genetically engineered cell contain the desired N-glycan. For example, at least about 27%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, or at least about 99% of the target proteins isolated from the genetically engineered cell can contain the desired N-glycan.

In some embodiments, the isolated modified target proteins can be frozen, lyophilized, or immobilized and stored under appropriate conditions, e.g., which allow the altered target proteins to retain biological activity.

Cultures of Engineered Cells

This document also provides a substantially pure culture of any of the genetically engineered cells described herein. As used herein, a “substantially pure culture” of a genetically engineered cell is a culture of that cell in which less than about 40% (i.e., less than about: 35%; 30%; 25%; 20%; 15%; 10%; 5%; 2%; 1%; 0.5%; 0.25%; 0.1%; 0.01%; 0.001%; 0.0001%; or even less) of the total number of viable cells in the culture are viable cells other than the genetically engineered cell, e.g., bacterial, fungal (including yeast), mycoplasmal, or protozoan cells. The term “about” in this context means that the relevant percentage can be 15% percent of the specified percentage above or below the specified percentage. Thus, for example, about 20% can be 17% to 23%. Such a culture of genetically engineered cells includes the cells and a growth, storage, or transport medium. Media can be liquid, semi-solid (e.g., gelatinous media), or frozen. The culture includes the cells growing in the liquid or in/on the semi-solid medium or being stored or transported in a storage or transport medium, including a frozen storage or transport medium. The cultures are in a culture vessel or storage vessel or substrate (e.g., a culture dish, flask, or tube or a storage vial or tube).

The genetically engineered cells described herein can be stored, for example, as frozen cell suspensions, e.g., in buffer containing a cryoprotectant such as glycerol or sucrose, as lyophilized cells. Alternatively, they can be stored, for example, as dried cell preparations obtained, e.g., by fluidized bed drying or spray drying, or any other suitable drying method.

The following are examples of the practice of the invention. They are not to be construed as limiting the scope of the invention in any way.

### EXAMPLES

### Example 1

### Introduction of Antibody Genes into Yarrowia lipolytica

The amino acid sequences for the anti-HER2 antibody heavy and light chains were obtained from Carter et al., Proc Natl Acad Sci USA, 89(10): 4285-4289 (1992); and Ward et al., Appl Environ Microbiol., 70(5): 2567-2576 (2004). The relevant amino acid sequences were reverse translated, codon-optimized for Yarrowia lipolytica, and synthesized by GenArt, Regensburg Germany. Regions of very high (>80%) or very low (<30%) GC content were avoided where possible. During the optimization processes, the following cis-acting sequence motifs also were avoided: internal TATA-boxes, chi-sites and ribosomal entry sites, AT-rich or GC-rich sequence stretches, repeat sequences and RNA secondary structures as well as (cryptic) splice donor and acceptor sites.

In order to allow secretion of the proteins, the coding sequence of the Lip2 protein ‘prepro’ signal (followed by that of a peptide linker ‘GGG’) was added to the 5′ region of the coding sequence for each of the light chain and heavy chains. A CACA enhancer element also was added 5′ to the start codon (ATG) for each of the light and heavy chain coding sequences. The resulting construct encoding the light chain was 769 nucleotides in length, and contained the following domains organized 5′ to 3′: the cacaATGprepro signal, the variable region (V_{L}), and the constant region (CO. The nucleotide sequence of the light chain (LC) construct is presented in FIG. 1A (SEQ ID NO:1). The encoded LC protein is 251 amino acids in length and approximately 25 kDa. FIG. 1C presents the amino acid sequence of the LC, with the LIP2 prepro leader sequence underlined, the V_{L} domain sequence underlined with two lines (V_{L} domain); and the Ck1 domain underlined with a dashed line (Ck1 domain).

The resulting construct encoding the heavy chain (HC) was 1482 nucleotides in length, and contained the following domains organized 5′ to 3′: the cacaATGprepro signal, the variable region (V_{H}) and three constant regions (C_{H}1-3). The “hinge” region straddled C_{H}1 and C_{H}2. The nucleotide sequence of the heavy chain construct is presented in FIG. 1A (SEQ ID NO:2). The encoded heavy chain protein is 486 amino acids in length and approximately 55 kDa. FIG. 1D presents the amino acid sequence of the HC, with the LIP2 prepro leader sequence underlined, the V_{H} domain sequence underlined with two lines (V_{H} domain); and the CH domain underlined with a dashed line (CH domain).

The construct encoding the HER2 light chain and the construct encoding the HE2 heavy chain each were cloned into a pJME vector, as BamHI/AvrII fragments, utilizing the URA3 or LIP2 locus, for targeted integration into the Y. lipolytica genome, and called pJME927PTLipUra3exPOX2 preproHerHC or pJME923PTUraLeu2ExPOX2 preproHerLC. No transposon elements were used. The pJME plasmid is a shuttle vector capable of replication in either E. coli or Y. lipolytica, and contains both bacterial and Y. lipolytica specific sequences. The bacterial portion of the plasmid is derived from the plasmid pHSS6, and includes a bacterial origin of replication (ori) and the kanamycin-resistant gene conferring resistance to kanamycin (KanR). The integration cassette portion of the plasmid contained a selectable marker gene (e.g., LEU2 or URA3) and an expression cassette composed of an hp4d or POX2 promoter and a multiple cloning site (MCS) to insert the αHER2 light chain or heavy chain coding sequence in frame with the terminator of LIP2 gene. The plasmids were digested with NotI to release the integration cassette before transformation of Y. lipolytica cells.

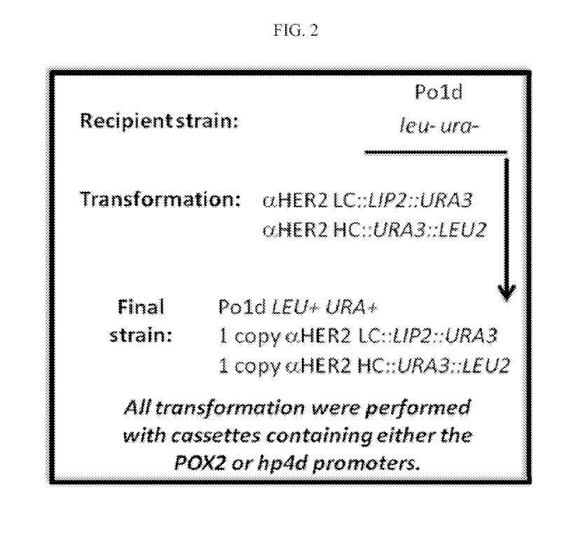

The NotI-digested heavy chain expression plasmid was introduced into Y. lipolytica strain Pold ((MatA ura3-302 leu2-270 xpr2-322). The integration of the heavy chain expression cassette into the URA3 locus was verified through Southern analysis. To construct a strain expressing the whole antibody, the NotI-digested light chain was introduced into the heavy chain expressing strain. Again, integration of the light chain expression cassette into the LIP2 locus was verified by Southern analysis. FIG. 2 depicts the strain genealogy.

### Example 2

### Identification of the Cleavage Site of the Antibody

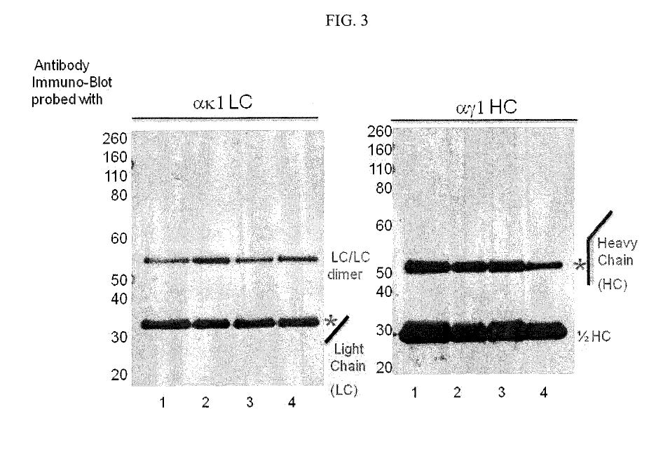

Transformants positive for both the heavy chain and light chain plasmids were cultured in SuperT rich medium for 96 h. The supernatant from the culture of four different clones was harvested and subjected to Western blot analysis. The light chain was detected using a monoclonal anti-human Kappa free light chain antibody (4C11) produced in a mouse (Product #1939, Abcam®). The heavy chain was detected using a monoclonal anti-human IgG (gamma chain specific) antibody produced in mouse (Product #15885 from Sigma). The light chain was present at the correct molecular weight (25 kDa) but exhibited a tendency to dimerize. Heavy chain also was detected at the correct molecular weight (50 kDa), but the majority was present as a degraded product with a molecular weight of approximately 32 kDa. See FIG. 3.

To identify the degradation site of the heavy chain produced by Y. lipolytica Pold cells, the heavy chain products were purified using protein G chromatography and subjected to N-terminal peptide sequencing. This revealed that the major antibody cleavage occurs at the Lys-Lys bond in the C_{H}1-hinge region.

### Example 3

### Construction of the Single Yapsin Knockout Strains of Yarrowia lipolytica

To determine if yapsin proteases were responsible for the degradation of the heavy chain, single yapsin knockout Y. lipolytica strains were produced. The sequences of the following yapsin3-like genes of Y. lipolytica (Yl) were obtained from the National Center for Biotechnology database (world wide web at.ncbi.nlm.nih.gov):

YPS1: YALI0E10175g Gene ID: 2912589, which encodes pYPS1

YPS2: YALI0E22374g Gene ID: 2912981, which encodes pYPS2

YPS3: YALI0E20823g Gene ID: 2911836, which encodes pYPS3

YPS4: YALI0D10835g Gene ID: 2910442, which encodes pYPS4

YPS5: YALI0A16819g Gene ID: 2906333, which encodes pYPS5

YPSX: YALI0C10135g Gene ID: 7009445, which encodes pYPSX

YPS7: YALI0E24981g Gene ID: 2912672, which encodes pYPS7

YPSXp: YALI0E34331g Gene ID: 2912367, which encodes pYPSXp

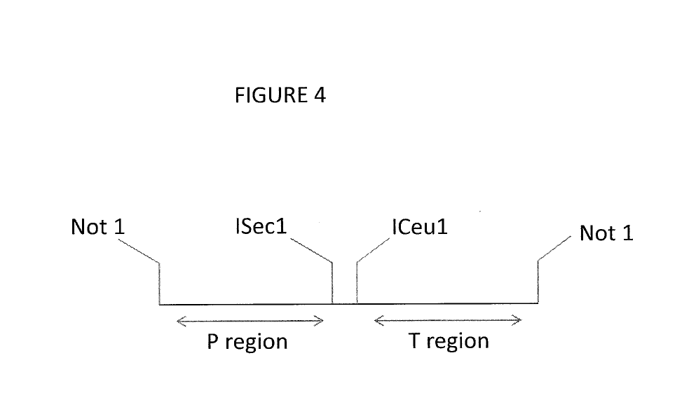

The promoter (“P”) and terminator (“T) regions flanking each yapsin open reading frame (ORF) target sequence were amplified using pairs of primers to obtain P and T fragments. The P and T fragments then were amplified using primer pairs that included unique cloning (restriction) sites (ISce1 and ICeu1), and which allowed the P and T fragments to be fused during a subsequent PCR and then cloned into the NotI E. coli moiety of an OXYP plasmid. Thus, each final disruption construct (cassette) contained NotI restriction sites at each end and the fusion region of the P and T fragments included the above mentioned two cloning (restriction) sites, one for insertion of a Y. lipolytica marker, and one for insertion of a promoter operably linked to a gene of interest so that the disruption constructs could also be used as targeted integration constructs. The disruption construct is depicted diagrammatically in FIG. 4.

Each yapsin disruption cassette was independently transformed into the Y. lipolytica pold antibody-expressing strain described above. Disruption of the locus was verified by Southern blot or PCR analysis. Single yapsin deleted strains were obtained for yps1, yps2, yps3, yps4, yps5, and yps7.

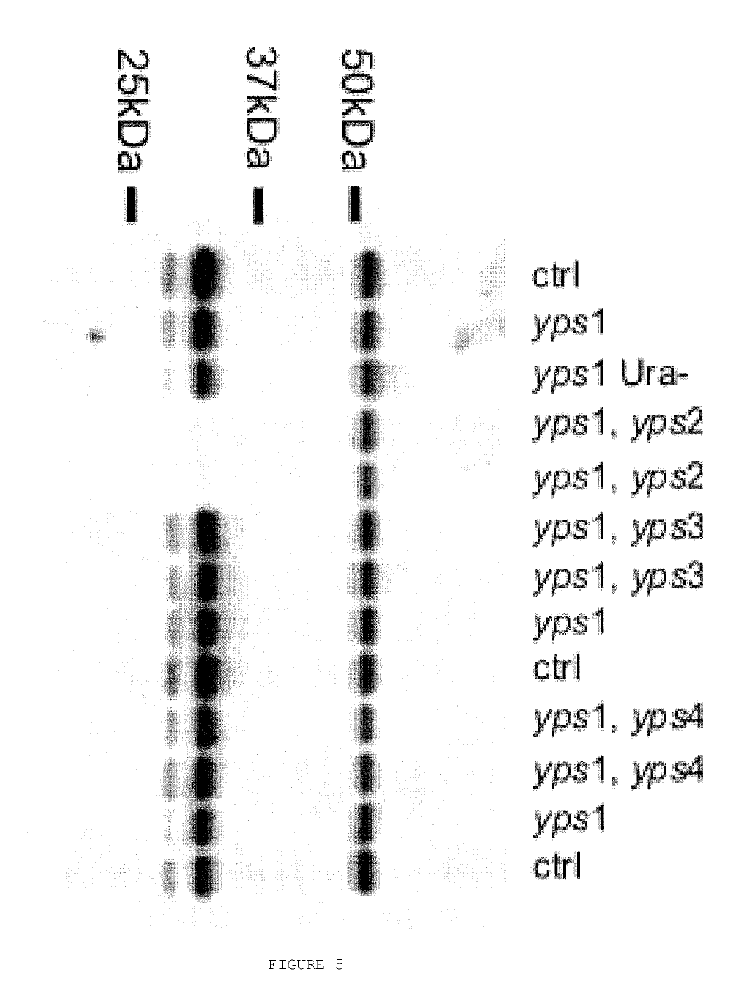

Unique clones representing individual disruptants, as well as a non-yapsin deleted control strain (ctrl), were grown separately in SuperT rich medium in a shakeflask. Culture supernatant samples were taken at 48 and 96 hours post inoculum and subjected to Western blot analysis to assess heavy chain degradation using a gamma chain specific anti-human IgG antibody produced in mouse (Sigma, 15885, monoclonal anti-human IgG). No cross reactivity was observed with the light chain. For the Δyps2, Δyps3, Δyps5, Δyps7, and Δypsx deletion strains, no reduction in proteolytic degradation was observed at 48 or 96 hours relative to control. See upper panel of FIG. 5.

For the Δyps1 and Δyps4 deletion strains, two clones of each strain were grown in SuperT medium in a shakeflask and culture supernatant samples were taken at 96 hours post inoculum and assessed for heavy chain degradation relative to the control strain. For the Δyps1 strains, a reduced amount of the 32 kDa breakdown product was detected as compared with both the Δyps4 and control strains, although extensive degradation remained. See lower panel of FIG. 5.

To further assess degradation in the Δyps1 deletion strain, the strain was grown in superT medium and culture supernatant samples were taken at 24 h, 40 h, 48 h, 60 h, 72 h and 96 h post inoculums and heavy chain degradation assessed relative to the control strain. For all timepoints, a reduction of the 32 kDa proteolytic product was observed compared to the control strain. At later timepoints, more degradation product was detected but still remained at a lower level than the degradation observed in control strains. These results indicate that there was a partial reduction of the proteolytic activity in the Δyps1 strain.

To determine if the disruption of two yapsin genes could further reduce proteolysis, a second yapsin gene was disrupted in the Δyps1 background. The following four strains were produced: Δyps1Δyps2, Δyps1Δyps3, Δyps1Δyps4 and Δyps1Δyps7. Correct disruption of the genes was verified by Southern analysis.

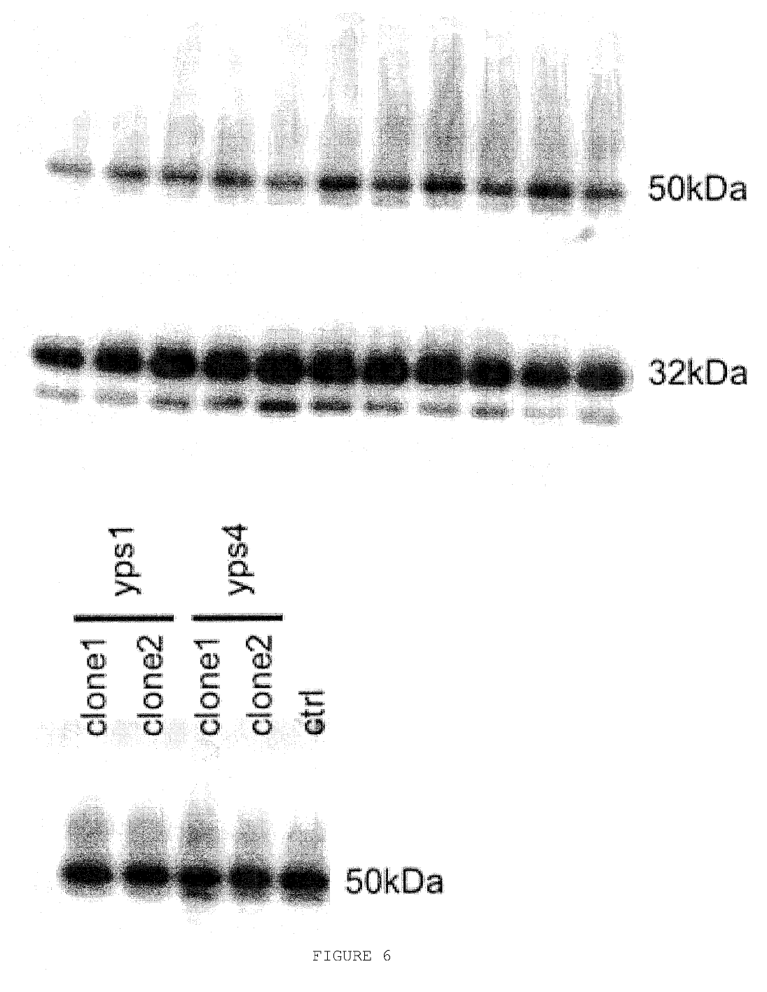

The Δyps1Δyps2, Δyps1Δyps3, and Δyps1Δyps4 strains and control strains (non-yapsin deletion, Δyps1, and Δyps1 URA-auxotrophic) were cultured. Supernatant samples were taken 96 hours post-inoculum and subjected to Western blotting. As shown in FIG. 6, no heavy chain degradation products were observed in the Δyps3.1Δyps3.2 strain. However, no overall increase in the amount of the full-length heavy chain product (50 kDa) was observed. In the Δyps1Δyps3, Δyps1Δyps4, and control strains, heavy chain degradation products were detected.

The amount of active secreted antibody was determined in the Δyps1Δyps2 strain and compared to that of the non disrupted strain via ELISA. No increase in total functional secreted product was detected.

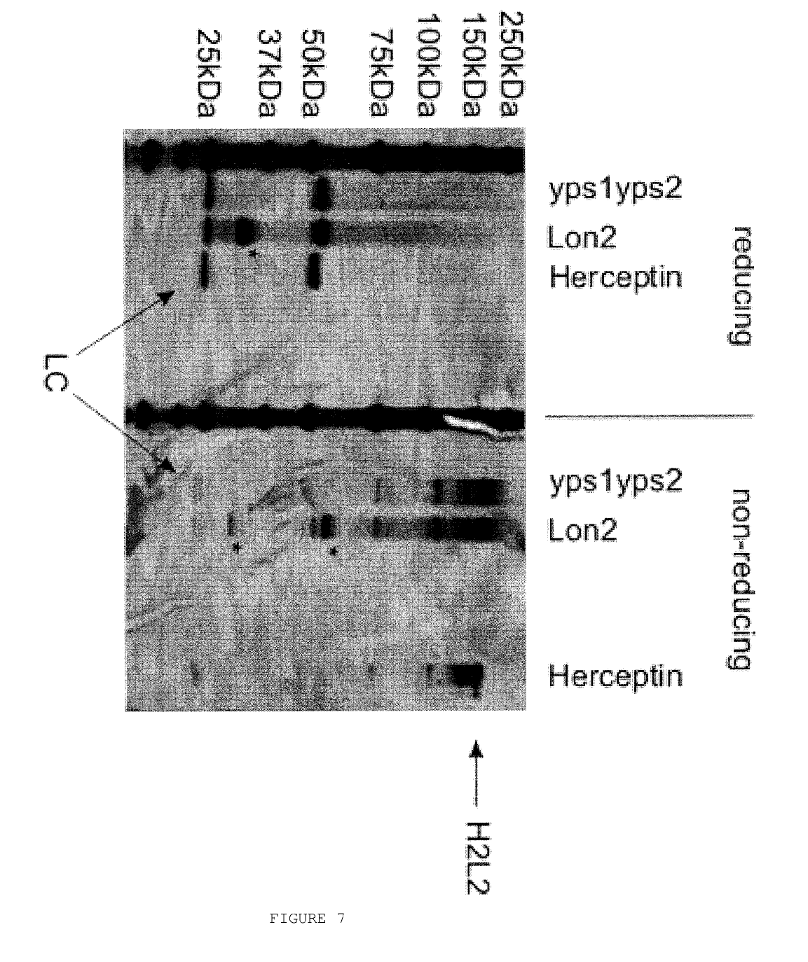

Protein G purified antibody derived from a Δyps1Δyps2 strain showed complete absence of heavy chain degradation products on a silver stained SDS-PAGE gel. See FIG. 7.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An isolated Yarrowia cell genetically engineered to comprise a deficiency in pYPS1 (YPS1 protein) activity and a deficiency in pYPS2 (YPS2 protein) activity.

2. The cell of claim 1, wherein said cell is a Yarrowia lipolytica cell.

3. The cell of claim 1, wherein said cell further comprises a nucleic acid encoding a target protein.

4. The cell of claim 3, wherein said target protein is a lysosomal protein, a pathogen protein, a growth factor, a cytokine, a chemokine, one or two polypeptide chains of an antibody or antigen-binding fragment thereof, or a fusion protein.

5. The cell of claim 4, wherein said antibody is selected from the group consisting of an antibody that binds vascular endothelial growth factor (VEGF), an antibody that binds to epidermal growth factor receptor (EGFR), an antibody that binds to CD3, an antibody that binds to tumor necrosis factor (TNF), an antibody that binds to TNF receptor, an antibody that binds to CD20, an antibody that binds to glycoprotein IIa/IIb receptor, an antibody that binds to IL2-receptor, an antibody that binds to CD52, an antibody that binds to CD11a, and an antibody that binds to HER2.

6. The cell of claim 4, wherein said antigen-binding fragment is selected from the group consisting of Fab, F(ab′)₂, Fv, and single chain Fv (scFv) fragments.

7. The cell of claim 1, wherein said cell is further deficient in OCH1 activity.

8. The cell of claim 1, wherein said cell comprises a nucleic acid encoding an alpha-1,2 mannosidase

9. The cell of claim 8, wherein said alpha-1,2 mannosidase comprises a targeting sequence to target said alpha-1,2 mannosidase to an intracellular compartment.

10. The cell of claim 1, wherein said cell is further deficient in ALG3 activity.

11. The cell of claim 1, wherein said cell further comprises a nucleic acid encoding an alpha-1,3-glucosyltransferase.

12. The cell of claim 1, said cell further comprising a nucleic acid encoding the alpha and beta subunits of a glucosidase.

13. The cell of claim 1, wherein said cell comprises a nucleic acid encoding a GlcNAc-transferase I.

14. The cell of claim 13, wherein said GlcNAc-transferase I comprises a targeting sequence to target said GlcNAc-transferase I to an intracellular compartment.

15. The cell of claim 1, wherein said cell comprises a nucleic acid encoding a GlcNAc-transferase II.

16. The cell of claim 15, wherein said GlcNAc-transferase II comprises a targeting sequence to target said GlcNAc-transferase II to an intracellular compartment.

17. The cell of claim 1, said cell further comprising a nucleic acid encoding a galactosyltransferase.

18. The cell of claim 17, wherein said galactosyltransferase comprises a targeting sequence to target said galactosyltransferase to the Golgi apparatus.

19. The cell of claim 1, wherein said cell does not produce detectable levels of a functional pYPS1 or a functional pYPS2.

20. The cell of claim 1, wherein said cell does not produce detectable mRNA molecules encoding functional pYPS1 and functional pYPS2.

21. The cell of claim 1, wherein the YPS1 and YPS2 genes are disrupted in the cell.

22. The cell of claim 1, wherein the YPS1 and YPS2 open reading frames are deleted.

23. A substantially pure culture of Yarrowia lipolytica cells, a substantial number of which are genetically engineered to comprise a deficiency in pYPS1 activity and a deficiency in pYPS2 activity.

24. An isolated Yarrowia cell genetically engineered to comprise (i) a deficiency in pYPS1 activity and (ii) a deficiency in pYPS2 activity, and one or more of (iii) a deficiency in ALG3 activity, (iv) a deficiency in OCH1 activity, (v) a nucleic acid encoding an alpha-1,2 mannosidase, (vi) a nucleic acid encoding a GlcNAc-transferase I, (vii) a nucleic acid encoding a GlcNAc-transferase II, (viii) a nucleic acid encoding a mannosidase II, (ix) a nucleic acid encoding an α-1,3-glucosyltransferase, (x) a nucleic acid encoding a galactosyltransferase, and (xi) a nucleic acid encoding the α and β subunits of a glucosidase.

25. The cell of claim 24, wherein said cell further comprises a nucleic acid encoding a target protein.

26. A method for reducing degradation of a target protein produced in Yarrowia, said method comprising expressing a nucleic acid encoding said target protein in a Yarrowia cell of claim 1.

27. A method for producing a target protein, said method comprising a) providing a Yarrowia cell genetically engineered to comprise a deficiency in pYPS1 activity, a deficiency in pYPS2 activity, and a nucleic acid encoding said target protein; and b) culturing said cell under conditions such that said cell produces said target protein.
